(19) United States
(12) Patent Application Publication
(10) Pub. No.: US 2022/0296515 A1
(43) Pub. Date: Sep. 22, 2022

(54) Vancomycin Liposome Compositions and Methods

(71) Applicant: Nevakar Injectables Inc., Bridgewater, NJ (US)

(72) Inventors: Karthik Yadav Janga, Bridgewater, NJ (US); Tushar Hingorani, Bridgewater, NJ (US); Jack Martin Lipman, West Milford, NJ (US); Kumaresh Soppimath, Skillman, NJ (US)

(73) Assignee: Nevakar Injectables Inc., Bridgewater, NJ (US)

(21) Appl. No.: 17/612,551

(22) PCT Filed: May 22, 2020
(86) PCT No.: PCT/US2020/034359

§ 371 Date: Nov. 18, 2021

Related U.S. Application Data
(60) Provisional application No. 62/853,597, filed on May 28, 2019.

(51) Int. Cl. A61K 9/127; A61K 38/14; A61K 9/00

(52) U.S. Cl. CPC A61K 9/1271; A61K 38/14; A61K 9/0019

(57) ABSTRACT

The inventive subject matter is directed to compositions and methods for liposomal vancomycin that have improved pharmacokinetics and enhanced drug loading and solution stability.

This application claims priority to our copending U.S. provisional patent application Ser. No. 62/853,597 filed May 28, 2019, which is incorporated by reference herein.

### FIELD OF THE INVENTION

The field of the invention is pharmaceutical compositions comprising vancomycin in a liposomal formulation, especially as it relates to injectable vancomycin compositions with improved pharmacokinetics, drug loading, and maintenance of high drug loading during processing.

### BACKGROUND

The background description includes information that may be useful in understanding the present invention. It is not an admission that any of the information provided herein is prior art or relevant to the presently claimed invention, or that any publication specifically or implicitly referenced is prior art.

Vancomycin is a branched tricyclic glycosylated non-ribosomal peptide antibiotic and is frequently used in the prophylaxis and treatment of infections caused by a variety of Gram-positive bacteria (and especially multi drug-resistant Staphylococcus aureus) that have failed to respond to conventional antibiotics.

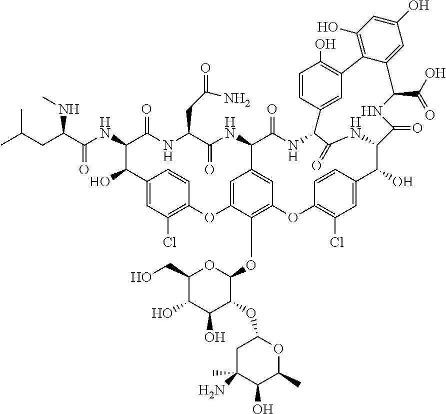 [SMILES: CN[C@H](CC(C)C)C(=O)N[C@H]1C(=O)N[C@@H](CC(N)=O)C(=O)N[C@H]2C(=O)N[C@H]3C(=O)N[C@H](C(=O)N[C@H](C(=O)O)c4cc(O)cc(O)c4-c4cc3ccc4O)[C@H](O)c3ccc(c(Cl)c3)Oc3cc2cc(c3O[C@@H]2O[C@H](CO)[C@@H](O)[C@H](O)[C@H]2O[C@H]2C[C@](C)(N)[C@H](O)[C@H](C)O2)Oc2ccc(cc2Cl)[C@H]1O]

Vancomycin

As vancomycin is a large glycopeptide with a molecular weight of ˜1450 Da, it is not appreciably absorbed via the oral route and thus administered intravenously. Vancomycin is administered at a relatively slow rate (e.g., over at least 1 hour) to avoid various adverse events, particularly thrombophlebitis and pain. In humans with normal renal function the half-life of Vancomycin is approximately three to six hours. It is eliminated primarily via the renal route, with >80%-90% recovered unchanged in urine within 24 h after administration of a single dose.

Vancomycin is still the antibiotic of choice for the treatment of methicillin resistant staphylococcus aureus (MRSA). The usual dose of Vancomycin is 2 grams divided as either 500 mg every six hours or 1 gram every 12 hours. After multiple intravenous infusions, 1 gram of Vancomycin infused over 60 minutes produces a mean plasma concentration of 8 μg/mL, 11 hours after the end of infusion in humans. To improve clinical outcomes for patients affected by MRSA, the plasma levels are recommended to be between 15 μg/mL to 20 μg/mL before the next dose is administered. Due to increased dose that needs to be administered to achieve higher terminal plasma concentrations, the incidence of vancomycin associated nephrotoxicity will unfortunately increase.

Various drugs can be formulated in liposomes to so enhance pharmacokinetics and pharmacodynamics. Conceptually, vancomycin when encapsulated in liposomes, can reduce the exposure of the drug to kidney, and reduced exposure to kidney would presumably reduce vancomycin associated nephrotoxicity. However, a significant challenge in encapsulating Vancomycin is the large dose that needs to be administered. Moreover, although Vancomycin has previously been encapsulated in liposomes, drug loading reported in the literature has been relatively low. In this context it should be appreciated that low drug loading of Vancomycin will lead to large quantities of lipids that are being administered to the patient, that in turn overloads the macrophage system of the patient.

For example, it is known to encapsulate polar small hydrophilic drugs into liposomes as is described in EP 1757270. All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. In the compositions of the '270 application, liposomes were formulated to comprise at least one neutral saturated phospholipid and at least one charged saturated lipid. However, such formulations are typically limited to small molecules such as 5-FU, and large molecules such as vancomycin will often have very low loading parameters. Moreover, the circulation time of most conventional liposomes is in many instances still relatively low.

Blood circulation time of liposomes can be increased by PEGylation, which may also increase vancomycin concentrations in target tissues lung and macrophages. In one example, PEGylated liposomal vancomycin was proposed to improve the efficacy of treatment of MRSA pneumonia (Antimicrobial Agents And Chemotherapy, Oct. 2011, p. 4537-4542). Here, vancomycin liposomes were initially prepared using a thin-film hydration method and an ammonium sulfate gradient method, which provided poor encapsulation efficiency and poor stability of the prepared formulations. Subsequent formulations were prepared using 1,2-distearoyl-sn-glycero-3-phosphocholine (DSPC), cholesterol, and polyethylene glycol (PEG) in 3:1:0 and 3:1:0.02 molar ratios in a modified dehydration-rehydration method. Unfortunately, loading and stability were less than desirable. In addition, the pharmacokinetic data for PEGylated and non-PEGylated liposomes did not reveal substantial differences. US 9566238 shows similar data.

In still other approaches, vancomycin hydrochloride liposomes (VANH-Lips) were prepared by a modified reverse phase evaporation method, and chitosan wrapped vancomycin hydrochloride liposomes (c-VANH-Lips) nanosuspensions were formulated by electrostatic deposition (International Journal of Pharmaceutics 495 (2015) 508-515). Unfortunately, such liposomal preparations also had relatively low encapsulation efficiency and drug loading. Yet further aspects and examples of PEGylated liposome compositions are described elsewhere (International Journal of Nanomedicine 2006:1(3) 297-315), with most of them suffer from low drug loading and/or solution instability.

Significantly higher vancomycin concentrations were disclosed in US 2009/0104257 where the liposomes had a relatively low lipid to drug ratio (e.g., 3:1 or less). Here, liposomes were prepared using a solvent injection process. While such method yielded liposomes with improved drug loading, the solvent stability of the liposomes was less than desirable. Indeed, agglomeration and phase separation were typically associated with the liposomal formulations of the '257 application.

Therefore, while various vancomycin compositions are known in the art, there is still a need for vancomycin compositions, preferably liposomal vancomycin compositions that are suitable for injection and that exhibit desirable pharmacokinetics and drug loading and have desirable solution stability.

### SUMMARY OF THE INVENTION

The inventive subject matter is directed liposomal vancomycin compositions and methods therefor that are suitable for injection and that exhibit desirable pharmacokinetics and drug loading.

In one aspect of the inventive subject matter, the inventors contemplate vancomycin liposome composition that comprises a plurality of liposomes encapsulating vancomycin, wherein the liposomes are disposed in aqueous solution that includes an osmolarity adjusting agent. In contemplated compositions the liposomes comprise a first lipid component, an optional second lipid component, cholesterol, and a PEGylated diglyceride, wherein the first lipid component comprises a C14:0 fatty acid portion and wherein the second lipid component comprises a C16:0 fatty acid portion.

In some embodiments, the liposomes have a particle size of 240 nm+/−15 nm at D₅₀, and/or the aqueous solution has a pH of equal or less than pH 5.5. It is further contemplated that the osmolarity adjusting agent is a non-ionic agent such as sucrose.

In further embodiments, the first and/or the second lipid component comprises a phosphatidyl choline portion. For example, a suitable first lipid components is 1,2-dimyristoyl-sn-glycero-3-phosphocholine, and/or a suitable second lipid component is 1,2-dipalmitoyl-sn-glycero-3-phosphocholine. The PEGylated diglyceride will preferably have a PEG chain with a molecular weight of 2,000+/−200, and/or include at least one C14:0 fatty acid portion (e.g., 1,2-dimyristoyl-rac-glycero-3-methylpolyoxyethylene). Therefore, it is contemplated that the liposomes may comprise the first and the second lipid component.

In still further embodiments, the weight ratio of the first and second lipid component to cholesterol is between 2.2-3.2:1 and 1.1:1, and/or the ratio of the first and second lipid component to the PEGylated diglyceride is between 11.4-16.6:1 and 5.6:1. Moreover, the vancomycin is present in contemplated compositions at a concentration of between 0.1-100 mg/ml (e.g., 5-6 mg/ml). Additionally, contemplated liposomes may have a drug loading of at least 0.55 mg or at least 0.80 mg vancomycin per mg of total lipid, and it is generally preferred that the composition is formulated for injection. As such, the composition will have an ethanol concentration of equal or less than 0.05% (v/v).

Therefore, and viewed from a different perspective, contemplated vancomycin liposome compositions may comprise or will essentially consist of a plurality of liposomes encapsulating vancomycin, wherein the liposomes are disposed in aqueous solution that includes an osmolarity adjusting agent. For example, such liposomes may comprise 1,2-dimyristoyl-sn-glycero-3-phosphocholine as a first lipid component, 1,2-dipalmitoyl-sn-glycero-3-phosphocholine as an optional second lipid component, a cholesterol, and 1,2-dimyristoyl-rac-glycero-3-methylpolyoxyethylene as a PEGylated diglyceride. Typically, but not necessarily, the liposomes have a particle size of 240 nm+/−15 nm at D₅₀ (or 400-450 nm +/−15 nm at D₉₀), and/or the aqueous solution has a pH of equal or less than pH 5.5. It is further preferred that the osmolarity adjusting agent is a non-ionic agent such as sucrose.

In contemplated liposomes the 1,2-dimyristoyl-rac-glycero-3-methylpolyoxyethylene may have a PEG chain with a molecular weight of 2,000+/−200, may have a weight ratio of the first and second lipid component to cholesterol of between 2.2-3.2:1 and 1.1:1, and/or the ratio of the first and second lipid component to the PEGylated diglyceride is between 11.4-16.6:1 and 5.6:1. Typically, vancomycin is present in the composition at a concentration of between 1-10 mg/ml, wherein the composition is preferably formulated for injection.

In another aspect of the inventive subject matter, the inventors also contemplate a method of producing a vancomycin liposome composition that includes a step of preparing an alcoholic lipid solution that comprises a first lipid component, an optional second lipid component, a cholesterol, and a PEGylated diglyceride, and a further step of preparing an aqueous vancomycin solution. In yet another step, the alcoholic lipid solution and the aqueous vancomycin solution are mixed in a microfluidics channel having a static mixer at a flow rate that is sufficient to form a product that comprises a plurality of liposomes encapsulating the vancomycin. In yet a further step, the product is subjected to tangential flow filtration or dialysis to remove the alcohol and non-encapsulated vancomycin.

In some embodiments, the tangential flow filtration or dialysis is performed with an aqueous solution comprising an osmolarity adjusting agent (e.g., sucrose), and/or the aqueous solution has a pH of equal or less than pH 5.5. Most typically, the liposomes have a particle size of 240 nm+/−15 nm at D₅₀.

In further embodiments, the first and/or the second lipid component comprise a phosphatidyl choline portion. For example, the first lipid component may be 1,2-dimyristoyl-sn-glycero-3-phosphocholine, the second lipid component may be 1,2-dipalmitoyl-sn-glycero-3-phosphocholine, the PEGylated diglyceride may comprise a PEG chain with a molecular weight of 2,000+/−200, and/or the PEGylated diglyceride comprises at least one C14:0 fatty acid portion (e.g., 1,2-dimyristoyl-rac-glycero-3-methylpolyoxyethylene).

Additionally, it is contemplated that the liposomes will comprise the first and the second lipid component, that the weight ratio of the first and second lipid component to cholesterol is between 2.2-3.2:1 and 1.1:1, and/or the ratio of the first and second lipid component to the PEGylated diglyceride is between 11.4-16.6:1 and 5.6:1. Where desired, the alcoholic lipid solution comprises ethanol.

In still other aspects of the inventive subject matter, the inventors contemplate a method of reducing nephrotoxicity of a vancomycin formulation that includes a step of encapsulating the vancomycin into liposomes, wherein the liposomes comprise a first lipid component, an optional second lipid component, a cholesterol, and a PEGylated diglyceride. In some embodiments the first lipid component comprises a C14:0 fatty acid portion (e.g., 1,2-dimyristoyl-sn-glycero-3-phosphocholine) and the second lipid component comprises a C16:0 fatty acid portion (e.g., 1,2-dipalmitoyl-sn-glycero-3-phosphocholine). In further embodiments, the liposomes have a particle size of 240 nm+/−15 nm at D₅₀, the PEGylated diglyceride is 1,2-dimyristoyl-rac-glycero-3-methylpolyoxyethylene, and/or the PEG chain in the PEGylated diglyceride has a molecular weight of 2,000+/−200.

It is contemplated that the reduced nephrotoxicity can be measured by reduction of a urinary biomarker that is indicative of nephrotoxicity as compared to administration of non-liposomal vancomycin in the same quantity. For example, reduced nephrotoxicity may be a reduction by at least 10%, or by at least 30%, or by at least 50% of a measured value of the urinary biomarker. Suitable biomarkers include KIM-1 and clusterin. Alternatively, or additionally, reduced nephrotoxicity may also be determined using a histopathological marker, such as tubular cell injury.

In still further aspects of the inventive subject matter, the inventors contemplate a method of increasing a pharmacokinetic parameter of vancomycin that includes a step of encapsulating the vancomycin into liposomes, wherein the liposomes comprise a first lipid component, an optional second lipid component, a cholesterol, and a PEGylated diglyceride. In some embodiments the first lipid component comprises a C14:0 fatty acid portion (e.g., 1,2-dimyristoyl-sn-glycero-3-phosphocholine) and the second lipid component comprises a C16:0 fatty acid portion (e.g., 1,2-dipalmitoyl-sn-glycero-3-phosphocholine). In further embodiments, the liposomes have a particle size of 240 nm+/−15 nm at D₅₀, the PEGylated diglyceride is 1,2-dimyristoyl-rac-glycero-3-methylpolyoxyethylene, and/or the PEG chain in the PEGylated diglyceride has a molecular weight of 2,000+/−200.

For example, Cₘₐₓ may be increased at least 5-fold, or at least 10-fold, AUC may be increased at least 30-fold, or at least 60-fold, and/or T_{1/2} may be increased at least 2-fold, or at least 4-fold.

Various objects, features, aspects and advantages of the inventive subject matter will become more apparent from the following detailed description of preferred embodiments, along with the accompanying drawing.

### BRIEF DESCRIPTION OF THE DRAWINGS

FIG. 1 is a graph depicting exemplary results for the impact of sucrose concentration on the liposome particle size.

FIG. 2 is a graph depicting exemplary results for the impact of the pH of a 3.42% sucrose concentration on the liposome particle size.

FIG. 3 is a graph depicting exemplary results for drug leakage from liposomes according to the inventive subject matter.

FIG. 4 is a graph depicting one set of exemplary results of selected pharmacokinetic parameters using liposomes according to the inventive subject matter.

FIG. 5 is a graph depicting another set of exemplary results of selected pharmacokinetic parameters using liposomes according to the inventive subject matter.

FIG. 6 is a schematic representation of a test procedure for the evaluation of nephrotoxicity.

FIG. 7 is a graph depicting selected pharmacokinetic results.

FIG. 8 is a graph depicting selected results for markers of kidney damage.

### DETAILED DESCRIPTION OF THE INVENTION

The inventive subject matter is directed to liposomal vancomycin compositions that are suitable for injection and that exhibit desirable pharmacokinetics, drug loading, and solution stability. Moreover, the inventors have also discovered that vancomycin liposomes can be prepared in a conceptually simple yet effective passive loading approach with high drug loading/entrapment.

More specifically, and as is described in more detail below, the inventors discovered that vancomycin liposomes can be prepared from one or more lipid component having relatively short fatty acid chain portions in combination with cholesterol and a PEGylated diglyceride. Notably, such liposomes exhibited not only advantageous drug loading parameters and solution stability, but could also be prepared via scalable manufacturing process such as microfluidics technology.

For example, the inventors prepared a vancomycin liposome composition in a microfluidics device by mixing (1) an alcoholic lipid solution that comprises a first lipid component, an optional second lipid component, a cholesterol, and a PEGylated diglyceride with (2) an aqueous vancomycin solution under conditions that formed a product comprising a plurality of liposomes encapsulating vancomycin. The product was then subjected to tangential flow filtration (TFF) or dialysis to remove alcohol and non-encapsulated vancomycin.

With respect to the first and/or the second lipid components it is contemplated that many lipid components suitable for liposomes are deemed appropriate for use herein, however, it is generally preferred that the first and/or the second lipid components will comprise a phosphatidyl choline portion. For example, and among other suitable choices, the first and/or second lipid component is 1,2-dimyristoyl-sn-glycero-3-phosphocholine and/or 1,2-dipalmitoyl-sn-glycero-3-phosphocholine. Moreover, the first lipid component may comprise a C12, C14, and/or C16 fatty acid portion. Likewise, the second lipid component may comprise a C14, C16, and/or C18 fatty acid portion. As will be readily appreciated, the ratio between first and second lipid components can vary considerably.

However, it is typically preferred that the first lipid component will include a C-14 fatty acid portion (typically esterified with the glycerol portion), and most preferably two C-14 fatty acid portions. Moreover, it is preferred (but not required) that one or both fatty acid portions will be saturated fatty acids. Alternatively, and especially where more flexibility is desired, one or both fatty acid portions may have one, or two, or three double bonds.

Where a second lipid component is present, the second lipid component will preferably include a C-16 fatty acid portion (typically esterified with the glycerol portion), and most preferably two C-16 fatty acid portions. As before, it is preferred (but not required) that one or both fatty acid portions in the second lipid component will be saturated fatty acids. Alternatively, and especially where more flexibility is desired, one or both fatty acid portions may have one, or two, or three double bonds. In other aspects, the second lipid component may also include a single or two C-18 or longer fatty acid portion (typically esterified with the glycerol portion). These longer fatty acids may have any degree of desaturation and so include one, two, three or more double bonds (which may be conjugated, cis- or trans-orientation).

Therefore, suitable lipid components in contemplated liposomes include one or more phosphatidyl cholines (PCs), phosphatidyl-glycerols (PGs), phosphatidic acids (PAs), phosphatidylinositols (PIs), phosphatidyl serines (PSs), and all reasonable mixtures thereof. For example, suitable lipid components may be egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and all reasonable combinations thereof

Particularly preferred lipid components in contemplated liposomes include 1,2-dimyristroyl-sn-glycero-3-phosphocholine, 1,2-dilauroyl-sn-glycero-3-phosphocholine, 1,2-distearoyl-sn-glycero-3-phosphocholine, 1,2-dimyristoyl-sn-glycero-3-phosphoethanolamine, 1,2-dipalmitoyl-sn-glycero-3-phosphoethanolamine, 1,2-dioleoyl-sn-glycero-3-phosphate monosodium salt, 1,2-dipalmitoyl-sn-glycero-3-[phosphor-rac-(1-glycerol)]sodium salt, 1,2-dimyristoyl-sn-glycero-3-[phospho-L-serine] sodium salt, 1,2-dioleoyl-sn-glycero-3-phosphoethanolamine-N-glutaryl sodium salt, 1,2-dipalmitoyl-sn-3-phosphatidylcholine, and 1,1′,2,2′-tetramyristoyl cardiolipin ammonium salt, and all reasonable combinations thereof

With respect to the PEGylated diglyceride it is typically preferred that the PEGylated diglyceride is a lipid compound as noted above in which at least one of the fatty acid portions is replaced or modified by a PEG portion. Most preferably, the PEG is a polyethylene glycol with an average molecular weight of about 500 to about 10,000 Daltons, which may optionally be substituted with an alkyl, alkoxy, acyl, or aryl moiety. For example, PEG may be substituted with a methyl at the terminal hydroxyl position. In another example, PEG will have an average molecular weight of about 750 to about 5,000 Daltons, more preferably, of about 1,000 to about 5,000 Daltons, and most preferably about 1,500 to about 3,000 Daltons. Among other suitable PEGylated diglycerides, those with C14 and/or C16 fatty acid portions are particularly preferred such as 1,2-dimyristoyl-rac-glycero-3-methylpolyoxyethylene (having a PEG chain with a molecular weight of 2,000+/−200).

Likewise, the cholesterol component may vary considerably. However, in most typical embodiments, the cholesterol component will be chemically unmodified cholesterol. Alternatively, the cholesterol may be chemically modified to include a butyrate portion or a phenylacetate portion, or a carbohydrate portion.

As will be readily appreciated, contemplated liposomes may comprise the first and/or the second lipid components, the PEGylated diglyceride, and/or the cholesterol component in various ratios. However, it is generally contemplated that cholesterol will be a minority component in the liposomes. Therefore, cholesterol (or any derivative thereof) may be present at equal or less than 50 mol %, or at equal or less than 40 mol %, or at equal or less than 30 mol %, or at equal or less than 20 mol %, or at equal or less than 15 mol %, or at equal or less than 10 mol %, or at equal or less than 5 mol %. Viewed form a different perspective, contemplated formulations may have a weight ratio of the first plus second lipid component to cholesterol of at least 2:1, or at least 2.5:1, or at least 3:1, or at least 3.5:1, or at least 4.0:1, or even higher. Therefore, exemplary weight ratios of the first plus second lipid component to cholesterol will be between 2.5:1 and 3.0:1, or between 3.0:1 and 3.5:1, or between 3.5:1 and 4.0:1. Similarly, it is noted that the PEGylated diglyceride component will generally be a minority component. For example, contemplated ratios of the first plus second lipid component to the PEGylated diglyceride may be between 5:1 and 10:1, or between 10:1 and 20:1, or between 20:1 and 30:1.

Moreover, it should be noted that while vancomycin is generally preferred, various other glycopeptide antibiotics are also deemed suitable for use herein. For example, glycopeptide antibiotics contemplated herein include, avoparcin, ristocetin, teicoplanin, and their derivatives, including vancomycin derivatives. For example, derivatives of vancomycin include multivalent vancomycins, PEGylated vancomycin conjugates, norvancomycin, vancomycin disulfides, synmonicin, mono- or di-dechlorovancomycin, glutamine analogs of vancomycin (e.g., A51568B, and M43G), aspartic acid analogs of vancomycin (e.g., M43F, M43B), desvancosamine derivatives of vancomycin (e.g., A51568A and M43A, and corresponding aglycones), chlorine derivatives of vancomycin (e.g., A82846B, A82846A (eremomycin), orienticin A, A82846C), benzylic amino sugar derivatives of vancomycin (e.g., A82846B), N-acyl vancomycins, N-aracyl vancomycins, N-alkyl vancomycins (such as octylbenzyl, octyloxybenzyl, butylbenzyl, butyloxybenzyl, and butyl, derivatives), or mixtures thereof.

Most preferably, contemplated liposome compositions will comprise vancomycin in an amount of at least 0.1 mg/ml, or at least 0.5 mg/ml, or at least 1.0 mg/ml, or at least 5.0 mg/ml, or at least 10 mg/ml, or at least 50 mg/ml, or at least 100 mg/ml, or even higher. For example, suitable compositions may comprise vancomycin in an amount of between 0.1 and 1 mg/ml, or between 1.0 and 3.0 mg/ml, or between 3.0 and 10 mg/ml, or between 10 and 50 mg/ml, or between 30 and 80 mg/ml or between 50 and 100 mg/l. Further exemplary compositions may thus include comprise vancomycin in an amount of between 0.1 and 0.5 mg/ml, or between 0.5 and 1 mg/ml, or between 1 and 3 mg/ml, or between 3 and 6 mg/ml, or between 5 and 7 mg/ml, or between and 7 and 9 mg/ml, or between 8 and 10 mg/ml, or between 10 and 15 mg/ml, or between 15 and 25 mg/ml, or between 25 and 50 mg/ml.

Suitable liposome compositions will comprise an aqueous liquid solution that is pharmaceutically acceptable for administration to a mammal. While preferred aqueous solutions will predominantly comprise or essentially consist of water, various water miscible co-solvents (e.g., short chain alcohols, small organic acids such as formic or acetic acid, DMF, DMSO, THF, NMP, etc.) are also deemed suitable for use herein. Most typically, such co-solvents will be present in an amount of equal or less than 15 wt %, or equal or less than 10 wt %, equal or less than 5 wt %, equal or less than 3 wt %, or equal or less than 1 wt %. Preferably, such liposomal solutions will contain non-encapsulated vancomycin in an amount of equal or less than 1 mg/ml, or equal or less than 0.5 mg/ml, or equal or less than 0.1 mg/ml, or equal or less than 0.01 mg/ml, and/or contain residual alcohol or other non-water solvent in an amount of equal or less than 1% v/v, or equal or less than 0.5% v/v, or equal or less than 0.1% v/v, or equal or less than 0.05% v/v. Most typically, suitable aqueous solutions will have pH that is equal or less than pH 5.5, or equal or less than pH 4.5, or equal or less than pH 3.5, equal or less than pH 3.0, or equal or less than pH 2.5. Viewed from another perspective, the pH of such solutions may be between 2.5-4.0, or between 3.0-5.0, or between 4.0 and 5.5. While not preferred, higher pH values are also contemplated.

It is further contemplated that the aqueous solution of the liposome composition will further comprise an osmolarity adjusting agent as also described in more detail below. With respect to suitable osmolarity adjusting agents it is contemplated that such agents may be non-ionic agent such as pharmaceutically acceptable sugars (e.g., various carbohydrate and carbohydrate derivatives), pharmaceutically acceptable salts, and various polar polymers that are known to increase tonicity. For example, suitable sugars include sucrose, mannitol, lactose, and dextrose, glucose, etc., while suitable salts include NaCl. The amount of tonicity adjusting agent used can be adjusted such that the osmolality of the liposome and surrounding fluid in the liposome composition are substantially matched (e.g., within 30 mOsm/kg, or within 20 mOsm/kg, or within 10 mOsm/kg). For example, the difference between the liposome and the surrounding fluid may be between 0.1-2 mOsm/kg, or between 2-5 mOsm/kg, or between 5-10 mOsm/kg, or between 10-20 mOsm/kg, or between 10-20 mOsm/kg, or between 20-3, 0 mOsm/kg. An osmometer can be used to check and adjust the amount of tonicity adjusting agent to be added to obtain the desired osmolality.

Where a buffer is included in contemplated liposome formulations, it is noted that suitable buffers are generally buffers that stabilize the pH of the contemplated formulations at or near a pH range in which vancomycin has a positive net charge, for example between pH 2.0 and 3.5, or between pH 3.5 and 4.0, or between pH 4.0 and 5.5. Therefore, the pH of contemplated formulations will be equal or less than 5.5, or equal or less than 4.0, or less than 3.5, or less than 3.0. For example, suitable compositions may have a pH of 2.3 (+/−0.2), or a pH of 2.5 (+/−0.2), or a pH of 2.7 (+/−0.2). While not limiting to the inventive subject matter, the buffer strength is typically relatively low, for example, equal or less than 100 mM, equal or less than 75 mM, equal or less than 60 mM, equal or less than 50 mM, or between 5 mM and 50 mM (e.g., 10 mM, 20mM, 30mM, 40 mM, or 50mM).

Therefore, in exemplary embodiments, the buffer is in the pharmaceutical composition in a concentration of from about 10 mM to about 75 mM, or from about 10 mM to about 60 mM, or from about 0.1 mM to about 60 mM, or from about 0.1 mM to about 55 mM, or from about 0.1 mM to about 50 mM, or from about 5 mM to about 60 mM, or from about 0.1 mM to about 10 mM, or from about 1 mM to about 10 mM, or from about 9 mM to about 20 mM, or from about 15 mM to about 25 mM, or from about 19 mM to about 29 mM, or from about 24 mM to about 34 mM, or from about 29 mM to about 39 mM, or from about 34 mM to about 44 mM, or from about 39 mM to about 49 mM, or from about 44 mM to about 54 mM, or from about 19 mM to about 54 mM, or from about 25 mM to about 54 mM. Of course, it should be appreciated that there are many types of buffer systems and buffers known in the art, and all of those are deemed suitable for use herein, including buffer systems comprising an acid and a salt of the acid, a first and a second salt (e.g., monobasic and dibasic salt), and amphoteric buffer molecules.

Moreover, in further contemplated aspects, the pharmaceutical composition may also include one or more chelating agents, and particularly metal ion chelators. For example, suitable chelators include various bicarboxylic acids, tricarboxylic acids, and aminopolycarboxylic acids such as ethylenediaminetetraacetic acid (EDTA), ethylene glycol-bis(β-aminoethyl ether)-N,N,N′,N′-tetraacetic acid (EGTA), and penta(carboxymethyl) diethylenetriamine (DTPA), and salts and hydrates thereof While not limiting to the inventive subject matter, it is contemplated that the metal ion chelators will slow down metal ion-catalyzed oxidation and microbial growth. For example, suitable chelator concentrations may be between 10 μg/ml and 50 μg/ml, between 50 μg/ml and 250 μg/ml, and between 100 μg/ml and 500 μg/ml. Viewed form a different perspective, chelator concentrations of equal or less than 0.03 wt %, or equal or less than 0.02 wt %, or equal or less than 0.01 wt % are contemplated.

Consequently, suitable chelating agents include monomeric polyacids such as EDTA, cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccmic acid (DMSA), aminotrimethylene phosphonic acid (ATPA), citric acid, ophthalmologically acceptable salts thereof, and combinations of any of the foregoing. Further suitable chelating agents include pyrophosphates, tripolyphosphates, and, hexametaphosphates, chelating antibiotics such as chloroquine and tetracycline, nitrogen-containing chelating agent containing two or more chelating nitrogen atoms within an imino group or in an aromatic ring (e.g., diimines, 2,2′-bipyridines, etc.), and various polyamines such as cyclam (1,4,7,11-tetraazacyclotetradecane), N-(C₁-C₃₀ alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), and deferoxamine (N′-[5-[[4-[[5-(acetylhydroxyamino)pentyl]amino]-1,4-dioxobutyl]hydroxy-amino]pentyl]-N′-(5-aminopentyl)-N-hydroxybutanediamide; also known as desferrioxamine B and DFO).

It should further be appreciated that contemplated liposome compositions may also include one or more preservatives. For example, preservatives that may be included are benzalkonium chloride, cetrimide or cetrimonium chloride or bromide, benzododecinium bromide, miramine, cetylpyridinium chloride, polidronium chloride or polyquaternium-1, polyquaternium-42 (also known as polixetonium), sepazonium chloride; mercurial derivatives such as the phenylmercury salts (acetate, borate or nitrate), mercuriothiolate sodium (otherwise called thiomersal or thimerosal) and mercurobutol; amidines such as chlorhexidine digluconate or polyhexamethylene biguanide (PHMB); alcohols such as chlorobutanol or phenylethanol or benzyl alcohol or phenol or m-cresol or phenoxyethanol; parabens or esters such as parahydroxybenzoic acid, methylparaben, and propylparaben). Most typically, the preservatives are added in an effective amount to reduce or avoid microbial growth. For example, preservatives may be present in the composition between 0.01-0.1 wt %, or between 0.05-0.5 wt %, or between 0.1-1.0 wt %.

In still further aspects of the inventive subject matter, it should be appreciated that most known liposome formation processes are deemed suitable for use herein, and contemplated processes include active and passive loading processes. Therefore, contemplated compositions can be prepared using various dry film hydration methods, spray drying processes, various solvent injection processes, etc. However, in particularly preferred aspects, the liposomes are formed in a microfluidics approach in which two solvents (one containing the lipid phase in an organic solvent and the other containing vancomycin in an aqueous phase) are fed in laminar flow to a mixing section, which preferably uses static mixing. An exemplary microfluidic device is known as NanoAssemblr™ (Precision Nanosystems Benchtop model, commercially available from Precision Nanosystems, 395 Oyster Point Boulevard, Suite 145 South San Francisco, Calif., 94080). In such methods, the lipids will preferably be provided in a solvent that will completely solubilize the lipids at the desired or needed concentration. For example, suitable solvents include various alcohols (and especially ethanol), chloroform, methylene chloride, hexane, cyclohexane, and all reasonable combinations thereof, etc., while the solvents that contain the vancomycin will especially include water, THF, DMF, DMSO, acetone, and all reasonable combinations thereof, etc.

As will be readily appreciated, the particular method of liposome formation will at least in part influence one or more process parameters, and especially drug loading/drug to lipid ratio, and entrapment efficiency. As is shown in more detail below, it is generally preferred that methods contemplated herein will provide a drug loading of at least 0.5 mg per mg of total lipids, or at least 0.6 mg per mg of total lipids, or at least 0.7 mg per mg of total lipids, or at least 0.8 mg per mg of total lipids, or at least 0.85 mg per mg of total lipids. Likewise, it is typically preferred that the processes contemplated herein will have a drug entrapment efficiency of at least 70%, or at least 75%, or at least 80%, or at least 85%, or at least 85.

Regarding the size distribution of the liposomes contemplated herein, it is typically preferred that the liposomes have an average particle size of equal of less than 800 nm, or equal of less than 600 nm, or equal of less than 500 nm, or equal of less than 400 nm, or equal of less than 300 nm, or equal of less than 200 nm. Viewed from a different perspective, the size distribution of the liposomes is preferably between 100-200 nm (e.g., 100+/−20 nm, or 120+/−20 nm, or 140+/−20 nm, or 160+/−20 nm, or 180+/−20 nm, or) at D₁₀, between 200-300 nm (e.g., 200+/−20 nm, or 220+/−20 nm, or 240+/−20 nm, or 260+/−20 nm, or 280+/−20 nm, or) at D₅₀, and/or between 400-500 nm (e.g., 400+/−20 nm, or 420+/−20 nm, or 440+/−20 nm, or 460+/−20 nm, or 480+/−20 nm, or) at D₉₀. Therefore, typical overall average particle sizes are about 150+/−20 nm, or about 175+/−20 nm, or about 200+/−20 nm, or about 225+/−20 nm, or about 250+/−20 nm, or about 275+/−20 nm, or about 300+/−20 nm, or about 325+/−20 nm, or about 350+/−20 nm.

Still further, and as shown in more detail below, the inventors particularly contemplate liposomes and liposome formulations with high drug-to-lipid ratios (e.g., at least 0.5 mg, or at least 0.6 mg, or at least 0.7 mg, or at least 0.8 mg of drug per mg of total lipids) that exhibit a substantial lack of agglomeration (e.g., less than 15% or less than 10% agglomerated after 4 weeks of storage at room temperature) and/or increase in particle size (e.g., less than 15% or less than 10% increase after 4 weeks of storage at room temperature), and/or that have substantially no loss of vancomycin due to drug leakage from the liposomes (e.g., less than 10% or less than 5% loss after 4 weeks of storage at room temperature). Most notably, and as shown in more detail below, these parameters were achieved by a combination of specific factors that individually would have resulted in a reduction of drug loading/entrapment efficiency, an increase in drug leakage, and an increase in agglomeration/size increase of liposomes.

With respect to the sterilization of contemplated formulations it should be appreciated that contemplated formulations may be sterilized using all known manners of sterilization, including filtration through 0.45 micron filters, heat sterilization, autoclaving, radiation (e.g., gamma, electron beam, microwave).

### EXAMPLES

The following examples illustrate some of the experiments leading to the formulations according to the inventive subject matter, however, should not be construed to limit the scope of the claims in any way.

Formulation Factors:

Unless indicated otherwise, liposomes were prepared using a microfluidic mixing process between an ethanolic lipid phase and aqueous phase carrying vancomycin using a NanoAssemblr™ platform (Precision Nanosystems Benchtop model, commercially available from Precision Nanosystems, 395 Oyster Point Boulevard, Suite 145 South San Francisco, Calif., 94080). Lipids were dissolved in ethanol as indicated below, and vancomycin was dissolved in water at approximately 150 mg/ml. Flow rates remained constant at 10 ml/min and mixing was performed in benchtop cartridges commercially supplied by Precision Nanosystems. The aqueous to organic ratio was 4:1. Temperature of the process was 35° C.

Due to the known difficulties to load liposomes with relatively large hydrophilic drugs, the inventors set out to investigate parameters that would impact various formulation factors such as drug loading, liposome stability, drug leakage from liposomes, etc. To that end, the inventors investigated the impact of different carbon chain lengths in lipids, and exemplary results are shown in Table 1 below.

As can be readily seen from the data, the vancomycin loading parameters decreased as the phospholipid chain length increased. In these experiments, the glass transition temperature (T_{g}) of the phospholipids was in the following rank order: C18>C16>C14. The lipid was added to ethanol and the temperature was increased over the glass transition temperature of the lipid. Vancomycin HCl solution at 150 mg/mL was also heated to the corresponding lipid temperature. The solutions were transferred in separate syringes and mixed in the microfluidic benchtop model to manufacture liposomes.

Drug loading was determined by determining the total drug amount of Vancomycin by an HPLC method versus unincorporated vancomycin. The liposomes were separated from the production fluid by centrifugation in a 100,000 molecular weight cut off centrifugal filter membrane available from EMD Millipore, and the filtrate was analyzed for the free (unincorporated) drug content. By subtracting the free drug from the total drug content, the encapsulated drug was determined. The ratio of encapsulated drug in mg to the theoretical lipid content of the solution is denoted herein as drug loading. Notably, the vancomycin loading in liposomes dramatically decreased with increasing T_{g} of lipids.

TABLE 1

| | Composition | | |
|---|---|---|---|
| Total Lipid | C-14 | | Drug |
| content | Phospholipid | Cholesterol | Loading |
| (mM) | (Mol %) | (Mol %) | (% w/w) |
| 0.34 | C-18 (85) | 15 | 9 |
| | C-16 (85) | 15 | 17 |
| | C-14 (85) | 15 | 160 |

Following the notable results of increased vancomycin loading at decreasing Tg, the inventors set out to further investigate whether or not the presence of other lipids would further impact drug loading. To that end, vancomycin liposomes were prepared using C-14 phospholipid components in combination with various other lipids and cholesterol. Total lipid concentration was also tested as a modifying factor on drug loading. Table 2 shows exemplary results for the impact of cholesterol, while Table 3 shows exemplary results for the impact of total lipid concentration.

TABLE 2

| | Composition | | |
|---|---|---|---|
| Total Lipid | Phospholipid | | Drug |
| content | chain length | Cholesterol | Loading |
| (mM) | (Mol %) | (Mol %) | (%) |
| 0.34 | 85 | 15 | 160 |
| | 70 | 30 | 128 |
| | 50 | 50 | 18 |

TABLE 3

| | Composition | | |
|---|---|---|---|
| Total Lipid | C-14 | | Drug |
| content | Phospholipid | Cholesterol | Loading |
| (mM) | (Mol %) | (Mol %) | (%) |
| 0.15 | 85 | 15 | 232 |
| 0.34 | | | 160 |
| 0.70 | | | 103 |
| 0.70 | 65 | 35 | 97 |

Interestingly, as the concentration of cholesterol increased vancomycin loading parameters sharply decreased. As can be seen from the results, vancomycin liposomes with 85 Mol % of lipid and 15 Mol % of cholesterol showed better vancomycin loading behavior. Moreover, the inventors observed that the impact of total lipid concentration on loading parameters was significant: As the total lipid composition increased, vancomycin loading parameters decreased.

In still further experiments, the inventors tested the impact of PEGylated lipids on loading parameters, and exemplary results are shown in Table 4. Here, C-14 saturated lipid PEGylated with PEG 2000 molecules were used to prepare PEGylated vancomycin liposomes. As can be seen from the results, vancomycin loading in the liposomes substantially reduced as PEGylated lipid concentrations increased.

TABLE 4

| | Composition | | | |
|---|---|---|---|---|
| Total Lipid | C-14 | DMG-PEG | | Drug |
| content | Phospholipid | 2000 | Cholesterol | Loading |
| (mM) | (Mol %) | (Mol %) | (Mol %) | (%) |
| 0.34 | 85 | — | 15 | 169 |
| | 84 | 1 | 15 | 135 |
| | 80 | 5 | 15 | 38 |

The impact of unsaturated lipids was further studied with respect to loading parameters, and exemplary results in Table 5 establish that unsaturated lipids led to reduced vancomycin loading.

TABLE 5

| | Composition | | | |
|---|---|---|---|---|
| | Phospholipid chain | | | |
| Total Lipid | length: Number of | DMG-PEG | | Drug |
| content | unsaturated bonds | 2000 | Cholesterol | Loading |
| (mM) | (Mol %) | (Mol %) | (Mol %) | (%) |
| 0.70 | 64 (C-14:0) | 1 | 35 | 85 |
| | 64 (C-18:1) | 1 | 35 | 76 |
| | 64 (C-22:1) | 1 | 35 | 65 |

In yet further experiments, the inventors also investigated the impact of combinations of C-14 (DMPC) and C-16 (DPPC) lipids. As can be seen from the data in Table 6 below, and at similar cholesterol levels, an increase in the C-16 lipid composition decreased the loading of vancomycin in liposomes. Remarkably, where C-14 phospholipids were used drug loading was relatively high even in the presence of PEGylated lipids (and even at higher concentrations of cholesterol).

TABLE 6

| | Composition | | | |
|---|---|---|---|---|
| Total Lipid | Mol % ratio of | DMG-PEG | | Drug |
| content | C-14:C-16 | 2000 | Cholesterol | Loading |
| (mM) | Phospholipids | (Mol %) | (Mol %) | (%) |
| 0.70-0.80 | 54:10 | 1 | 35 | 70 |
| | 44:20 | 1 | 35 | 72 |
| | 64:10 | 1 | 15 | 90 |
| | 54:30 | 1 | 15 | 81 |
| | 55:10 | — | 35 | 77 |
| | 45:20 | — | 35 | 67 |
| | 65:20 | — | 15 | 78 |
| | 55:30 | — | 15 | 64 |

Processing Factors:

The inventors studied various processing factors that can impact loading of vancomycin liposomes. Using conventional methods such as active loading of empty liposomes with vancomycin are unsatisfactory since active loading is more ideal for weakly basic molecules. Even if the drug is encapsulated by active loading, precipitation of Vancomycin within the liposomes is difficult due to the fact that vancomycin has a formal charge of zero, and as such cannot be precipitated as is commonly done with conventional liposome loading (e.g., via salt formation and/or pH). On the other hand, due to the large size of vancomycin, passive liposome loading is also difficult as can be seen from drug loading results in the heretofore known art.

In an effort to overcome the shortcomings of the conventional techniques when loading liposomes, the inventors explored liposome loading via microfluidic techniques. Here, vancomycin was loaded into liposomes by mixing an alcoholic lipid solution with an aqueous vancomycin solution in a microfluidic laminar flow device that included static mixing elements in the common flow channel. More specifically, a NanoAssemblr™ instrument was used with disposable cassettes at constant aqueous-to-organic fluid mixing ratio of 4:1 and at constant temperature (35° C.). In the exemplary data below, only the impact of flow rate on liposome formation and drug loading parameters was tested and typical results are shown in Tables 7 and 8.

TABLE 7

| Total | Composition | | | | |
|---|---|---|---|---|---|
| Lipid | C-14 | DMG-PEG | | | Drug |
| content | Phospolipid | 2000 | Cholesterol | Flow Rate | Loading |
| (mM) | (Mol %) | (Mol %) | (Mol %) | (mL/min) | (%) |
| 0.70 | 64 | 1 | 35 | 10 | 109 |
| | 64 | 1 | 35 | 15 | 146 |
| | 64 | 1 | 35 | 20 | 99 |

TABLE 8

| Total | Composition | | | | |
|---|---|---|---|---|---|
| Lipid | Mol % ratio of | DMG-PEG | | | Drug |
| content | C-14 & C-16 | 2000 | Cholesterol | Flow Rate | Loading |
| (mM) | Phospolipids | (Mol %) | (Mol %) | (mL/min) | (%) |
| 0.70 | 44 & 20 | 1 | 35 | 10 | 83 |
| | 44 & 20 | 1 | 35 | 15 | 111 |
| | 44 & 20 | 1 | 35 | 20 | 150 |

It should be noted, however, that while the data shown in the tables above indicated that increasing flow rates increased drug loading parameters, drug loading parameters declined in the formulation obtained after removal of free drug from the system as shown in more detail below, suggesting vancomycin adsorption on the surface of the liposomes during initial manufacturing processes.

In a further set of experiments, the inventors performed removal of free drug and ethanol from the liposomal formulation comparing two distinct processes, dialysis and tangential flow filtration. Exemplary results for these processes are shown in Tables 9 and 10, respectively. As can be readily seen, significant amount of free drug remained in the liposomes formulation even after 4 h washing. Significant drop in the drug loading parameters was noticed during dialysis process. Since molecular weight of vancomycin is high, it could not pass through 1 K and 10 K membranes. Tangential flow filtration (TFF) technique was adopted to remove free drug and ethanol from the vancomycin liposomes.

TABLE 9

| Total | Composition | | | | |
|---|---|---|---|---|---|
| Lipid | C-14 | | | | Drug |
| content | Phospolipid | Cholesterol | Membrane | Washing | Loading |
| (mM) | (Mol %) | (Mol %) | (MWCO) | Time (h) | (%) |
| 0.34 | 85 | 15 | 1K | 0 | 184 |
| | | | | 2 | 103 |
| | | | | 4 | 94 |
| | 85 | 15 | 10K | 0 | 184 |
| | | | | 2 | 95 |
| | | | | 4 | 32 |

TABLE 10

| Total | Composition | | | | |
|---|---|---|---|---|---|
| Lipid | C-14 | | | TFF | Drug |
| content | Phospolipid | Cholesterol | Membrane | Washing | Loading |
| (mM) | (Mol %) | (Mol %) | (MWCO) | cycles | (%) |
| 0.34-0.40 | 85 | 15 | 100K | 0 | 101 |
| | | | | 4 | 27 |

The TFF process was significantly faster than the dialysis process. However, the drug loading parameters reduced significantly, indicating leakage of drug from the liposomes during the process. Thus, while microfluidic passive loading of vancomycin into liposomes appeared to provide an attractive solution to problems associated with conventional vancomycin liposome formulations, surface adsorption and liposome stability seemed to be confounding factors to passive loading using microfluidic mixing.

In an effort to reduce surface adsorption and/or increase liposome stability, liposomes were prepared with an increased cholesterol composition. The results in Table 11 illustrate the impact of increased cholesterol in liposomes composition on the drug loading parameters during TFF (using 100 K MWCO membrane). Unexpectedly, an increase of cholesterol up to 30 mol % improved the loading parameters in the final liposomes formulation. At 50 mol % cholesterol, the drug loading decreased significantly. Unfortunately, however, all liposomes were settling down by the end of TFF process indicating agglomeration and/or an increase in particle size in the PBS buffer.

TABLE 11

| Total | Composition | | | TFF (100K | |
|---|---|---|---|---|---|
| Lipid | C-14 | | | MWCO) | Drug |
| content | Phospolipid | Cholesterol | TFF | Washing | Loading |
| (mM) | (Mol %) | (Mol %) | buffer | cycles | (%) |
| 0.34 | 70 | 30 | PBS | 0 | 51 |
| | | | | 4 | 68 |
| | 50 | 50 | | 0 | 42 |
| | | | | 4 | 22 |

Therefore, the inventors investigated the impact of water as a TFF buffer on the loading parameters and particle size, and exemplary results are shown in Table 12. Notably, while drug entrapment increased significantly in the liposomes by the end of TFF process, drug loading was reduced.

TABLE 12

| Total | Composition | | | TFF (100K | |
|---|---|---|---|---|---|
| Lipid | C-14 | | | MWCO) | Drug |
| content | Phospolipid | Cholesterol | TFF | Washing | Loading |
| (mM) | (Mol %) | (Mol %) | buffer | cycles | (%) |
| 0.34 | 70 | 30 | Water | 0 | 99 |
| | | | | 6 | 54 |

To further improve the drug entrapment, which indicates the removal of free drug, the total lipid content was doubled and tested for TFF (using 100 K MWCO membrane), with water as buffer and exemplary results are shown in Table 13. Unfortunately, liposomes were settling down during the TFF process indicating agglomeration and/or an increase in particle size in water.

TABLE 13

| Total | Ingredients | | | TFF (100K | |
|---|---|---|---|---|---|
| Lipid | C-14 | | | MWCO) | Drug |
| content | Phospolipid | Cholesterol | TFF | Washing | Loading |
| (mM) | (Mol %) | (Mol %) | buffer | cycles | (%) |
| | 65 | 35 | Water | 0 | 97 |
| 0.70 | | | | 4 | 115 |

The inventors then set out to test the impact of different TFF buffers on the drug loading parameters and liposomes settling and exemplary results are shown in Table 14. Remarkably, the loading parameters for liposomes in saline as TFF fluid increased significantly compared to those observed in the water: ˜80% drug entrapment suggested that only 20% of free drug was remaining in the final liposomes formulation. Unfortunately, the liposomes settled down during the TFF process indicating agglomeration and/or an increase in particle size in the saline solution.

TABLE 14

| Total | Composition | | | TFF (100K | |
|---|---|---|---|---|---|
| Lipid | C-14 | | | MWCO) | Drug |
| content | Phospolipid | Cholesterol | TFF | Washing | Loading |
| (mM) | (Mol %) | (Mol %) | buffer | cycles | (%) |
| 0.70 | 65 | 35 | Water | 0 | 97 |
| | | | | 4 | 115 |
| | | | Saline | 0 | 182 |
| | | | | 4 | 109 |

In an effort to reduce or avoid agglomeration and/or an increase in particle size, the inventors also investigated the impact of DMG-PEG 2000 on the liposomes settling. Remarkably, and as can be seen from the results in Table 15, inclusion of 1 mol % DMG-PEG 2000 in the liposomes did reduce the drug loading parameters to some degree but resulted in liposomes that were stable without settling for 3h after the TFF process, unlike the non-PEGylated liposomes which settled during the TFF process.

TABLE 15

| | Composition | | | | TFF (100 K | |
|---|---|---|---|---|---|---|
| Total Lipid | | DMG-PEG | | | MWCO) | Drug Loading |
| content (mM) | C-14 (Mol %) | 2000 (Mol %) | Cholesterol (Mol %) | TFF buffer | Washing cycles | (%) |
| 0.70-0.80 | 65 | — | 35 | Saline | 0 | 182 |
| | | | | | 4 | 109 |
| | 64 | 1 | 35 | | 0 | 116 |
| | | | | | 4 | 83 |

When analyzing particle sizes, the inventors noted that the particle size of the non-PEGylated liposomes was 1318 nm while the PEGylated liposomes were 197 nm. That result suggested that the non-PEGylated liposomes grew bigger during the TFF, which confirmed that the settling of liposomes was due to particle size growth. The inventors therefore hypothesized that particle size growth could be attributed to an osmolarity difference across the liposome bilayer membrane, particularly in liposomes having C14 and/or C16 fatty acid components in the membrane lipids.

Consequently, the inventors investigated the impact of osmolarity adjusting agents (here: sucrose) in the TFF buffer on liposomes settling. In the examples below, the pH and osmolarity of vancomycin HCl solution was 2.65 and 103 mOsmol, respectively. Therefore, a 3.42% w/v sucrose solution was prepared as TFF buffer to maintain 103 mOsmol across the bilayer membrane. Also, the pH of sucrose solution was adjusted to 2.65 to induce a positive charge on vancomycin. Notably, with such modifications, drug permeability across the bilayer membrane was very low, resulting in substantially reduced drug leakage during and/or after TFF. Exemplary results are shown in Table 16.

TABLE 16

| | Composition | | | | TFF (100 K | |
|---|---|---|---|---|---|---|
| Total Lipid | C-14 | DMG-PEG | | | MWCO) | Drug Loading |
| content (mM) | (Mol %) | 2000 (Mol %) | Cholesterol (Mol %) | TFF buffer | Washing cycles | (%) |
| 0.70-0.80 | 65 | — | 35 | 3.42% w/v | 5 | 161 |
| | 64 | 1 | 35 | Sucrose | 5 | 81 |
| | | | | solution (pH | | |
| | | | | 2.65) | | |

Still further, it was observed that non-PEGylated liposomes were stable without settling for about 4-5 h, but then settled down. In contrast, PEGylated liposomes did not settle down over a period of at least one week. Therefore, it should be appreciated that 1 mol % of DMG-PEG 2000 reduced the particle size growth of the liposomes. Moreover, the modified TFF process with adjusted osmolarity (e.g., 3.42% w/v sucrose solution) and adjusted pH (e.g., pH 2.65) was effective in removing the free drug and ethanol.

The impact of sucrose concentration was then tested on the drug loading parameters and particle size distribution of liposomes, and exemplary results are shown in Table 17. Here, the drug loading parameters were low at 1% w/v sucrose concentration, while these parameters did not change significantly between 3.42 — 7.5% w/v and the were higher at 10% w/v sucrose.

TABLE 17

| | Composition | | | TFF buffer (sucrose | TFF (100 K | |
|---|---|---|---|---|---|---|
| Total Lipid | | DMG-PEG | | concentration) % | MWCO) | Drug Loading |
| content (mM) | C-14 (Mol %) | 2000 (Mol %) | Cholesterol (Mol %) | w/v | Washing cycles | (%) |
| 0.70 | 64 | 1 | 35 | 1 | 0 | 85 |
| | | | | | 6 | 44 |
| | | | | 3.42 | 0 | 85 |
| | | | | | 6 | 83 |
| | | | | 7.5 | 0 | 80 |
| | | | | | 6 | 62 |
| | | | | 10 | 0 | 78 |
| | | | | | 6 | 80 |

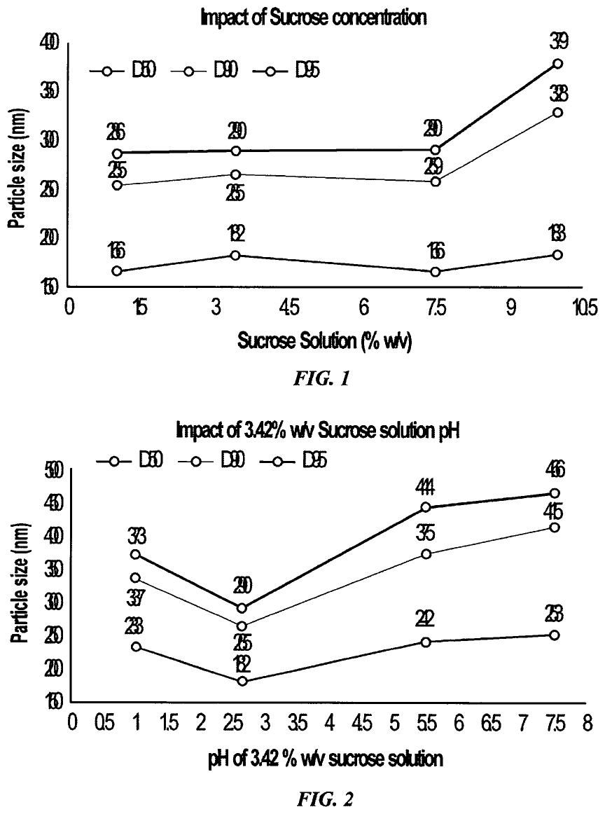

A similar trend was noticed in the particle size as can be seen in FIG. 1. The particle size distribution did not change between 1-7.5% w/v sucrose concentrations. The particle size was significantly higher at 10% w/v sucrose concertation and hence, the drug loading was higher. However, the drop in drug entrapment at this sucrose concentration indicates leakage of drug from the bigger liposomes.

In yet another experiment, the inventors tested the impact of 3.42% w/v sucrose buffer pH on drug loading parameters and particle size distribution, and exemplary results are shown in Table 18. Notably, no significant difference was noticed in the drug loading parameters by changing the sucrose buffer pH ranging from 1.0 to 5.5. However, the drug loading parameters dropped significantly at pH 7.5, especially after TFF process. Indeed, the following pKa values were noted for vancomycin: pKa₁=2.6; pKa₂=7.2; pKa₃=8.6; pKa₄=9.6; pKa₅=10.5; pKa6 =11.7. Consequently, the drop at pH7.5 could be attributed to adsorption of vancomycin, in unionized form at this pH, on the surface of liposomes. The particle size was lower at pH 2.65 compared to that observed at any other pH of the sucrose buffer as shown in FIG. 2.

TABLE 18

| | Ingredients | | | | TFF (100 K | |
|---|---|---|---|---|---|---|
| Total Lipid | C-14 | DMG-PEG | | pH of 3.42% w/v | MWCO) | Drug Loading |
| content (mM) | (Mol %) | 2000 (Mol %) | Cholesterol (Mol %) | sucrose buffer | Washing cycles | (%) |
| 0.70 | 64 | 1 | 35 | 1 | 0 | 90 |
| | | | | | 6 | 61 |
| | | | | 2.65 | 0 | 85 |
| | | | | | 6 | 83 |
| | | | | 5.5 | 0 | 107 |
| | | | | | 6 | 80 |
| | | | | 7.5 | 0 | 230 |
| | | | | | 6 | 23 |

Using the above data, liposomes with combinations of C-14 and C-16 lipids were prepared and subjected to TFF to optimize a formulation with better drug loading features and exemplary results are shown in Table 19.

TABLE 19

| | Ingredients | | | | TFF (100 K | |
|---|---|---|---|---|---|---|
| Total Lipid | C-14 & C-16 | DMG-PEG | | | MWCO) | Drug Loading |
| content (mM) | (Mol %) | 2000 (Mol %) | Cholesterol (Mol %) | TFF buffer | Washing cycles | (%) |
| 0.70 | 44 & 20 | 1 | 35 | 3.42% w/v | 0 | 113 |
| | | | | Sucrose | 5 | 69 |
| | 64 & 20 | 1 | 15 | solution (pH | 0 | 94 |
| | | | | 2.65) | 5 | 19 |
| | 55 & 10 | — | 35 | | 0 | 134 |
| | | | | | 5 | 89 |
| | 45 & 20 | — | 35 | | 0 | 119 |
| | | | | | 5 | 69 |

As can be seen from the data, the liposomes with 44 mol % of C-14 lipid and 20 mol % C-16 lipid with 35 mol % of cholesterol and 1 mol % DMG-PEG 2000 showed better loading parameter and particle size distribution compared to other compositions. On the basis of the above data and studies, two liposomes compositions — one with C-14 (64 mol %) lipid, cholesterol (35 mol %) and DMG-PEG 2000 (1 mol %), and one with a combination of C-14 (44 mol %) and C-16 (20 mol %) lipids, cholesterol (35 mol %) and DMG-PEG 2000 (1 mol %), showed higher drug loading parameters with desirable particle size distribution. Table 20 depicts exemplary vancomycin liposome compositions, which were further tested in additional in vitro and in vivo experiments.

TABLE 20

| S. No. | Ingredient | Liposomes 1 | Liposomes 2 |
|---|---|---|---|
| 1 | Vancomycin HCL | 5.1 mg/mL | 5.0 mg/mL |
| 2 | DMPC (C-14:0) | 3.087 mg/mL | 2.12 mg/mL |
| 3 | DPPC (C-16:0) | — | 1.047 mg/mL |
| 3 | DMG-PEG 2000 | 0.185 mg/mL | 0.185 mg/mL |
| 4 | Cholesterol | 0.96 mg/mL | 0.96 mg/mL |
| 5 | 3.42% Sucrose (pH 2.65) | 99.8 % w/v | 99.8% w/v |

The liposomes in the “Liposomes 1” compositions had a loading of 0.87 mg vancomycin per mg of total lipid, and the following particle size distribution: 143 nm (at D₁₀), 249 nm (at D₅₀), and 450 nm (at D₉₀). The liposomes in the “Liposomes 2” compositions had a loading of 0.56 mg vancomycin per mg of total lipid, and the following particle size distribution: 143 nm (at D₁₀), 249 nm (at D₅₀), and 450 nm (at D₉₀).

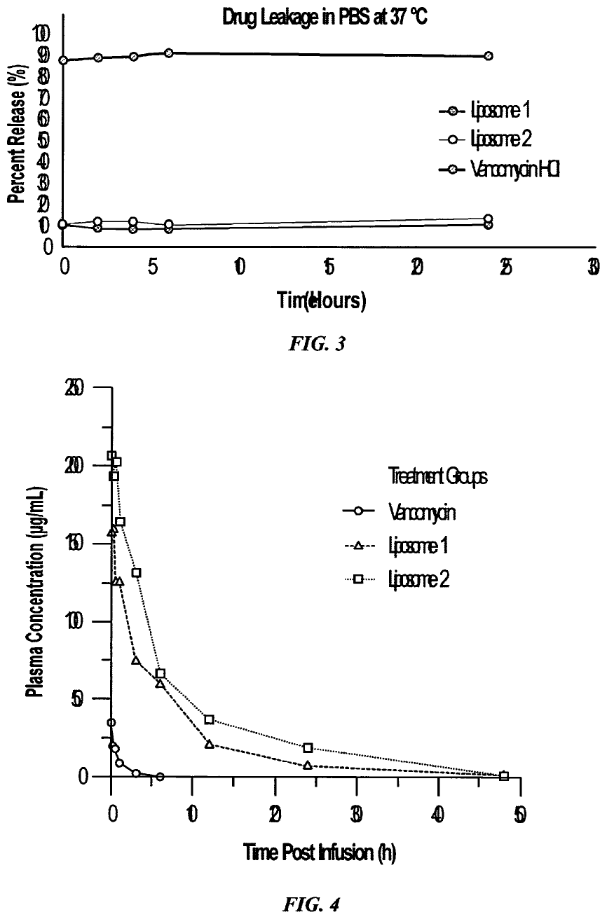

In vitro drug leakage behavior of the above two vancomycin liposomes compositions was tested, and FIG. 3 shows exemplary results. As is readily apparent, no significant drug leakage was observed over a period of at least 24 hours. Compared to control vancomycin solution, both liposomes formulations showed no apparent drug leakage over 24 h period in PBS, at 37° C. Additionally, both tested formulations were stable for week at 2-8° C. without any change in drug loading parameters and particle size distribution. The average particle size of liposomes in both compositions was around 230 nm.

Pharmacokinetic studies:

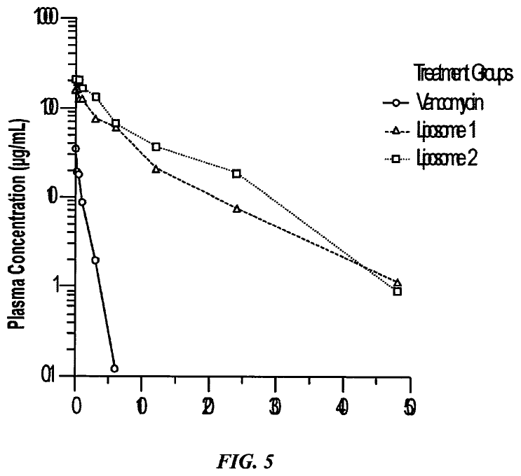

The two liposome formulations as described above were tested in rat models and exemplary results are shown in FIG. 4 and FIG . 5. As can be readily seen from the graphs and Table 21 below, the liposome formulations significantly increased serum half-life times, a significant increase in exposure and Cₘₐₓ was observed (FIG. 4, Table 21), and clearance was linear for all formulations, which is indicative of the renal route (FIG. 5). Thus, vancomycin was available for a prolonged period and less frequent administration is enabled.

TABLE 21

| | Dose | Infusion | | Cₘₐₓ | AUC_{0-∞} | | |
|---|---|---|---|---|---|---|---|
| Group | (mg/kg) | time (h) | T_{1/2} (h) | (μg/mL) | (μg * h/mL) | Tₘₐₓ (h) | Lambda z |
| Vancomycin | 15 | 1 | 0.8 | 34.6 | 34.8 | 0 | 0.879 |
| Liposome 1 | 15 | 1 | 8.6 | 159.2 | 1071.2 | 0.25 | 0.081 |
| Liposome 2 | 15 | 1 | 6.4 | 206.0 | 1659.9 | 0 | 0.109 |

Toxicity Studies:

To evaluate toxicity of the liposomal formulations, rats were dosed daily with 150 mg/kg test formulation for 5 days, which is about 10-fold of a dose for human administration (typically 1 g). For these experiments, Liposome 1 and Liposome 2 were used and had a composition as described in Table 20 above.

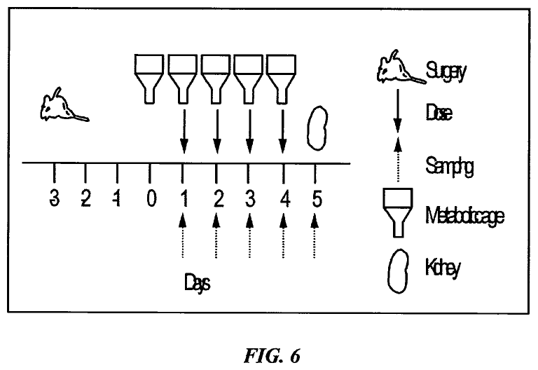

The endpoints for renal damage were urinary biomarkers (KIM-1, Clusterin, Osteopontin), a plasma biomarker (creatinine), and histopathology findings for the kidney. A general flow chart of the animal experiment is shown in FIG. 6. Here, blood sampling was performed at a total volume of 2 mL and clinical chemistry samples were drawn pre-dosing, day 3, and day 5 (0.25 mL each), and pharmacokinetic samples were drawn on Day 1 (4 samples of 0.125 mL), Day 3 (4 samples of 0.125 mL), and Day 5 (2 samples of 0.125 mL). For each animal, at euthanasia, kidneys were formalin fixed and flash frozen in liquid nitrogen. Samples were analyzed for histopathological grading using standard procedures well known in the art.

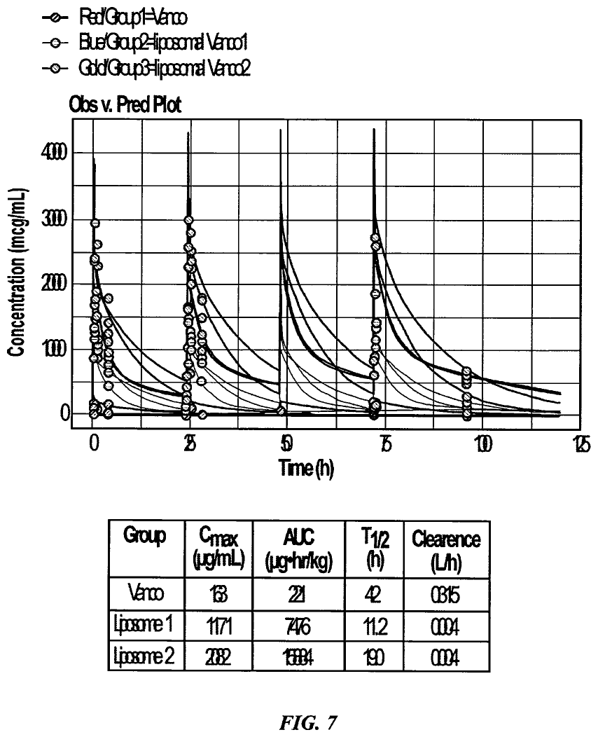

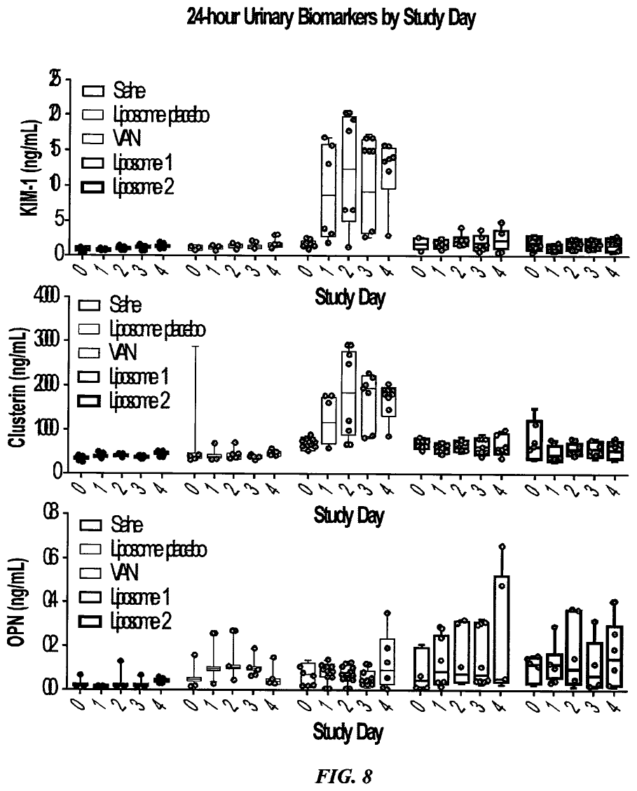

Exemplary pharmacokinetics results for daily dosing of vancomycin and selected formulations as presented herein are shown in FIG. 7. As can be readily seen from the graphs in FIG. 7, the liposomal preparations according to the inventive subject matter achieved a significantly higher Cₘₐₓ and AUC as compared to vancomycin administered per se. Still further, the liposomal formulations also exhibited substantially higher T_{1/2} and significantly reduced clearance. With further reference to FIG. 7, vancomycin half-life increased 2.7-4.5-fold with an 7-12-fold increase in Cₘₐₓ. Exposure of vancomycin (AUC) increased 33-71 times in liposomal formulations as compared to vancomycin administration alone, while the clearance of vancomycin from the systemic circulation decreased 78-fold.

Therefore, it is contemplated that when vancomycin is encapsulated into the liposomal formulations as presented herein, Cₘₐₓ may be increased at least 2-fold, at least 5-fold, at least 7-fold, or at least 10-fold, or even more, while exposure as measured by AUC may be increased at least 10-fold, at least 20-fold, at least 30-fold, at least 50-fold, or at least 60-fold, while T_{1/2} may be increased at least 1-fold, at least 2-fold, at least 3-fold, at least 4-fold, or at least 5-fold, or even more.

Further exemplary results are shown for selected urinary markers in FIG. 8. Here, the markers included KIM-1, clusterin, and osteopontin (OPN). As is readily evident from the graphs, there was no significant elevation of the markers in the saline control and empty liposome group, while the vancomycin treatment group exhibited significant elevations of all markers. On the other hand, the markers remained substantially unchanged or only moderately elevated over control in both treatment groups where liposomal compositions according to the inventive subject matter were used.

Indeed, based on these and other experiments (data not shown), the inventors contemplate that nephrotoxicity of a vancomycin formulation can be reduced by encapsulating the vancomycin in liposomes as presented herein. Thusly encapsulated vancomycin has remarkably reduced nephrotoxicity as compared to administration of equal quantities of non-liposomal vancomycin. For example, when nephrotoxicity was analyzed based on urinary biomarkers of nephrotoxicity, a dramatic decrease of these damage-associated biomarkers can be observed. For example, typical decreases (as measured by a percentage in reduction of the quantified marker) for urinary biomarkers are often at least 10%, or at least 20%, or at least 30%, or at least 40%, or at least 50%, or at least 60%, or at least 70%, or at least 80%, or at least 90%, or even higher. In some cases, no statistically significant difference will be observable against placebo (empty liposome) or vehicle control. Therefore, typical reductions in nephrotoxicity as measured by urinary biomarkers may be in the range of between 10-30%, or between 20-40%, or between 30-60%, or between 50-80%, or between 70-90%, and even higher.

These results were also mirrored in the histopathological findings for the various treatment groups. More particularly, in the group receiving vancomycin, significant proximal tubular cell injury and repair was observed that was consistent with vancomycin renal damage. On the other hand, in treatment groups receiving the Liposome formulations as presented herein (Liposome 1 and 2), glomerular alterations included mesangial expansion and intracapillary foam cells (lipid laden macrophages), which is consistent with renal lipid overload. The liposomal placebo group exhibited glomerular alterations, including foamy minimally-staining material in the urinary space of the glomerular capsule with or without mesangial expansion, while in the saline control no pathological changes were observed.

Therefore, and viewed form a different perspective, reduction of nephrotoxicity can be quantified by a reduction in the severity and incidence or frequency of one or more histopathological findings, such as tubular cell injury. Such reduction as compared to non-liposomal vancomycin control given at the same dosage may be at least 10%, or at least 20%, or at least 30%, or at least 40%, or at least 50%, or at least 60%, or at least 70%, or at least 80%, or at least 90%, or even higher. In some cases, no statistically significant difference will be observable against placebo (empty liposome) or vehicle control. Therefore, typical reductions in nephrotoxicity as measured by histopathological findings may be in the range of between 10-30%, or between 20-40%, or between 30-60%, or between 50-80%, or between 70-90%, and even higher.

Based on these results it should be appreciated that contemplated liposome formulations had a marked decrease in early biomarkers of proximal renal tubule damage due to vancomycin treatment, and that the liposomes encapsulation of vancomycin resulted in no observed histopathological changes in the kidneys due to vancomycin. Thus, it should be recognized that the liposomal formulations presented herein dramatically reduce vancomycin induced renal toxicity (>50% reduction in vancomycin induced proximal renal tubule damage).

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. Moreover, where the term ‘about’ is used in conjunction with a numeral, a range of that numeral+/−10%, inclusive, is contemplated. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

It should be apparent, however, to those skilled in the art that many more modifications besides those already described are possible without departing from the inventive concepts herein. The inventive subject matter, therefore, is not to be restricted except in the spirit of the disclosure. One skilled in the art will recognize many methods and materials similar or equivalent to those described herein, which could be used in the practice of the present invention. Indeed, the present invention is in no way limited to the methods and materials described.

Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

1. A vancomycin liposome composition, comprising:
a plurality of liposomes encapsulating vancomycin, wherein the liposomes are disposed in an aqueous solution, and wherein the composition is formulated for injection;
wherein the liposomes comprise a first lipid component, an optional second lipid component, a cholesterol, and a PEGylated diglyceride;
wherein the first lipid component comprises a C14:0 fatty acid portion and wherein the second lipid component comprises a C16:0 fatty acid portion, and
wherein the liposomes have a drug loading of at least 0.55 mg vancomycin per mg of total lipid and exhibit no apparent loss of vancomycin from the liposomes in PBS at 37° C. over a period 24 hours.

2. The liposome composition of claim 1, wherein the liposomes have a particle size of 240 nm+/−15 nm at D₅₀ and/or wherein the aqueous solution has a pH of equal or less than pH 5.5.

3. (canceled)

4. The liposome composition of claim 1, wherein the aqueous solution includes an osmolarity adjusting agent.

5. (canceled) .

6. The liposome composition of claim 1, wherein the first and/or the second lipid component comprises a phosphatidyl choline portion.

7. The liposome composition of claim 1, wherein the first lipid component is 1,2-dimyristoyl-sn-glycero-3-phosphocholine.

8. The liposome composition of claim 1, wherein the second lipid component is 1,2-dipalmitoyl-sn-glycero-3-phosphocholine.

9. The liposome composition of claim 1, wherein the PEGylated diglyceride comprises a PEG chain with a molecular weight of 2,000+/−200.

10. (canceled)

11. The liposome composition of claim 1, wherein the PEGylated diglyceride is 1,2-dimyristoyl-rac-glycero-3-methylpolyoxyethylene.

12. (canceled)

13. The liposome composition of claim 1, wherein the ratio of the first and second lipid component to cholesterol is between 3.0:1 and 3.4:1, and/or wherein the ratio of the first and second lipid component to the PEGylated diglyceride is between 10:1 and 20:1.

14. (canceled)

15. The liposome composition of claim 1, wherein the vancomycin is present in the composition at a concentration of between 1-10 mg/ml.

16. (canceled)

17. The liposome composition of claim 1, wherein the liposomes have a drug loading of at least 0.80 mg vancomycin per mg of total lipid.

18. (canceled)

19. The liposome composition of claim 1, wherein the composition has an ethanol concentration of equal or less than 0.05% (v/v).

20-29. (canceled)

30. A method of producing a vancomycin liposome composition, comprising:
preparing an alcoholic lipid solution that comprises a first lipid component, an optional second lipid component, a cholesterol, and a PEGylated diglyceride;
preparing an aqueous vancomycin solution;
mixing in a microfluidics channel having a static mixer the alcoholic lipid solution with the aqueous vancomycin solution at a flow rate that forms a product that comprises a plurality of liposomes encapsulating vancomycin;
subjecting the product to tangential flow filtration or dialysis to remove the alcohol and non-encapsulated vancomycin; and
wherein the liposomes have a drug loading of at least 0.55 mg vancomycin per mg of total lipid and exhibit no apparent loss of vancomycin from the liposomes in PBS at 37° C. over a period 24 hours.

31. The method of claim 30 wherein the step of tangential flow filtration or dialysis is performed with an aqueous solution comprising an osmolarity adjusting agent.

32. The method of claim 31 wherein osmolarity adjusting agent is sucrose,. and/or wherein the aqueous solution has a pH of equal or less than pH 5.5.

33. (canceled)

34. The method of claim 30 wherein the liposomes have a particle size of 240 nm+/−15 nm at D₅₀.

35. The method of claim 30 wherein the first and/or the second lipid component comprises a phosphatidyl choline portion.

36. The method of claim 30 wherein the first lipid component is 1,2-dimyristoyl-sn-glycero-3-phosphocholine and/or wherein the second lipid component is 1,2-dipalmitoyl-sn-glycero-3-phosphocholine.

37-39. (canceled)

40. The method of claim 30 wherein the PEGylated diglyceride is 1,2-dimyristoyl-rac-glycero-3-methylpolyoxyethylene.

41. (canceled)

42. The method of claim 30 wherein the ratio of the first and second lipid component to cholesterol is between 3.0:1 and 3.4:1, and/or wherein the ratio of the first and second lipid component to the PEGylated diglyceride is between 10:1 and 20:1.

43-65. (canceled)
